(19) United States
(12) Patent Application Publication
(10) Pub. No.: US 2012/0091181 A1
(43) Pub. Date: Apr. 19, 2012

(54) Apparatus and Method for Carrying and Storing Medical Drains

(76) Inventor: Margaret Barnes, Huntington Beach, CA (US)

(21) Appl. No.: 12/903,926

(22) Filed: Oct. 13, 2010

(51) Int. Cl. A45F 3/00; A45F 3/14; A61M 27/00

(52) U.S. Cl. 224/600; 604/541; 224/660

(57) ABSTRACT

A drain pouch for accommodating and carrying a plurality of medical drains that are inserted into a patient after a surgical procedure requiring the continuous removal of fluid away from a surgical site. The drain pouch may be comprised of lightweight, water resistant netting, or an opaque material fabric with flaps that effectively obscure the medical drains from view. The drain pouch may comprise a neck cord or a waist band that is worn about the patient, the plurality of medical drains being disposed within a corresponding plurality of pockets defined within the pouch. With the drain pouch secured about the patient, the patient is left unencumbered by the medical drains and maintains the use of both hands for everyday tasks, thus increasing the probability of patient treatment compliance and avoiding unnecessary possible added treatment time.

### BACKGROUND OF THE INVENTION

1. Field of the Invention

The invention relates to the field of disposable medical supplies, specifically to an apparatus and method for containing and carrying a plurality of medical drains associated with a medical procedure unobtrusively for the duration of a patient's treatment.

2. Description of the Prior Art

Medical drains comprised of latex, plastic or plastic composites, rubber, and other materials have long been used to drain fluids away from a cavity, wound, or surgical site. For example after a surgery, the patient is sent home with one or more drains implanted in their bodies for a period of time ranging from a few weeks to a few months. The patient is forced to carry the drains which are connected to the surgical site or internal organ via a series of tubing for the duration of their treatment. The drain may take the form of a Jackson-Pratt or “grenade” drain, or may be an ordinary medical receptacle bag as is known in the art. Depending on what is needed or what type of surgery has been recently performed, up to four drains may be used on a single patient, for example after a double mastectomy. In other cases, a single, larger bag may be required for other procedures such as the draining of an infected gallbladder.

No matter the type of drain used however, an incredible inconvenience is placed on the patient who must physically struggle with the drains while at the same time trying to maintain some form of normal lifestyle. Everyday tasks such as bathing or cooking become extremely difficult if not impossible without the help of a second person.

Several common sense solutions have been attempted to alleviate the burden on the patient such as pinning the drains to the outside of their clothes or attaching the drain to a chair or other object with a Velcro strap or series of ties. While these previous do help somewhat, they are not without their faults. Pinning the drains to the clothes of the patient can be problematic as brushing the drain against another object may cause the drain to detach from its tubing or break. Additionally, drains located on the outside of one's clothing do not provide a sense of normalcy that many patient's may desire, not to mention that the drains would have to be removed every time the patient wishes to bathe or remove their clothes. Temporarily coupling of the drains to a chair or other stationary object severely limits the mobility of the patient which may be critical if the patient requires physical therapy or other regular movement.

What is needed is an apparatus that permits a patient who requires one or more implanted medical drains to continue their normal lifestyle while at the same time helping to facilitate their compliance with their required treatment regimen. The apparatus should be simple to use, robust enough to accommodate multiple drains, and cost effective enough to manufacture so as to be disposable.

### BRIEF SUMMARY OF THE INVENTION

The illustrated embodiment of the invention include a portable drain pouch for holding at least one medical drain comprising at least one pocket for holding the corresponding at least one medical drain; a plurality of nylon tapes binding the plurality of pockets on three sides; and means for coupling the drain pouch to the patient, patient's clothing or object in proximity to the patient.

The pocket is comprised of water-resistant netting material.

The portable drain pouch of claim 1 where the plurality of nylon tapes binding the at least one pocket on three sides comprises a top nylon tape coupled to a top edge of the drain pouch.

The top nylon tape comprises an aperture and an internal passageway defined therethrough.

The means for coupling the drain pouch to the patient comprises a neck cord threaded through the internal passageway and the aperture at both ends of the top nylon tape, wherein the neck cord is adaptable to being formed into a loop and draped over the neck and/or shoulder of the patient.

The means for coupling the drain pouch to the patient comprises a belt threaded through the internal passageway and the aperture at both ends of the top nylon tape, wherein the belt is adaptable to being formed into a loop and wrapped around the waist of the patient.

The top nylon tape comprises a waist band forming a loop for wrapping around the waist of the patient, and wherein the waist band comprises a buckle for coupling both ends of the waist band together.

The means for coupling the drain pouch to the clothing of the patient, patient's clothing or object in proximity to the patient comprises a plurality of clips coupled to the top nylon tape.

The portable drain pouch of claim 1 where the at least one pocket is comprised of opaque material.

The portable drain pouch further includes at least one opaque flap disposed over the at least one pocket, wherein the flap conceals a top portion of the pocket when in a down position.

A portable apparatus for holding a plurality of medical drains comprising a plurality of pockets coupled to one another, a plurality of nylon tapes binding the plurality of pockets on three sides, and means for coupling the drain pouch to the patient or patient's clothing.

In one embodiment at least two of the plurality of pockets are coupled to one another in an abutting back-to-back configuration.

The plurality of nylon tapes binding the plurality of pockets comprises a top nylon tape disposed above the plurality of pockets, wherein the top nylon tape comprises an aperture and an internal hollow passageway defined therethrough.

The means for coupling the drain pouch to the patient comprises a neck cord threaded through the internal passageway and the apertures at both ends of the top nylon tape, wherein the neck cord is adapted to being formed into a loop and draped over the neck and/or shoulder of the patient.

The means for coupling the drain pouch to the patient comprises a belt threaded through the internal passageway and the apertures at both ends of the top nylon tape, wherein the belt is adapted to being formed into a loop and wrapped around the waist of the patient.

The plurality of nylon tapes binding the plurality of pockets comprises a top nylon tape disposed above the plurality of pockets, wherein the top nylon tape comprises a waist band forming a loop for wrapping around the waist of the patient, and wherein the waist band comprises a buckle for coupling both ends of the waist band together.

The illustrated embodiment of the invention also includes a method for accommodating and transporting a plurality of medical drains on or around a patient comprising the steps of affixing a drain pouch around or to the patient's person, placing a medical drain within at least one pocket defined within the drain pouch, and maintaining the medical drain within the at least one pocket defined within the drain pouch for the duration of the patient's treatment.

The step of affixing the drain pouch around or to the patient's person comprises the step of wrapping a neck cord coupled to the drain pouch about the patient's neck and/or shoulders.

The step of affixing the drain pouch around or to the patient's person comprises the step of wrapping a waist band coupled to the drain pouch about the patient's waist.

The method further includes the step of concealing the medical drain from view after placing it within the at least one pocket defined within the drain pouch.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a plan view of the front and back of a first embodiment of the drain pouch.

FIG. 2 is a diagram illustrating some of the fabrication steps of one of the embodiments.

FIG. 3 is a diagram illustrating the configuration of the pouch in one of the fabrication steps of one of the embodiments.

FIG. 4 is top plan view of the netting of the pouch disposed in a butterfly configuration dividing it into a forward portion and a rear portion.

FIG. 5 is a diagram of the assembled pouch shown carried over the shoulder of a patient.

FIG. 6 is a plan view of an embodiment wherein a neck protector is included.

FIG. 7 is a plan view of an embodiment of a pouch provided with a clip and swivel.

FIG. 8 is a perspective view of an embodiment shown with an opened flap and double drain pouch.

FIG. 9 is a perspective view of an embodiment shown with as a waist mounted pouch.

FIG. 10 is a perspective view of an embodiment shown with a neck cord and a pair of straps.

FIG. 11 is a plan view of an embodiment shown where the pockets of the pouch are in a stacked or tiered configuration.

FIG. 12 is a diagram illustrating the fabrication steps for a top pocket of the pouch seen in FIG. 11.

FIG. 13 is a diagram illustrating the fabrication steps for a bottom pocket of the pouch seen in FIG. 11.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

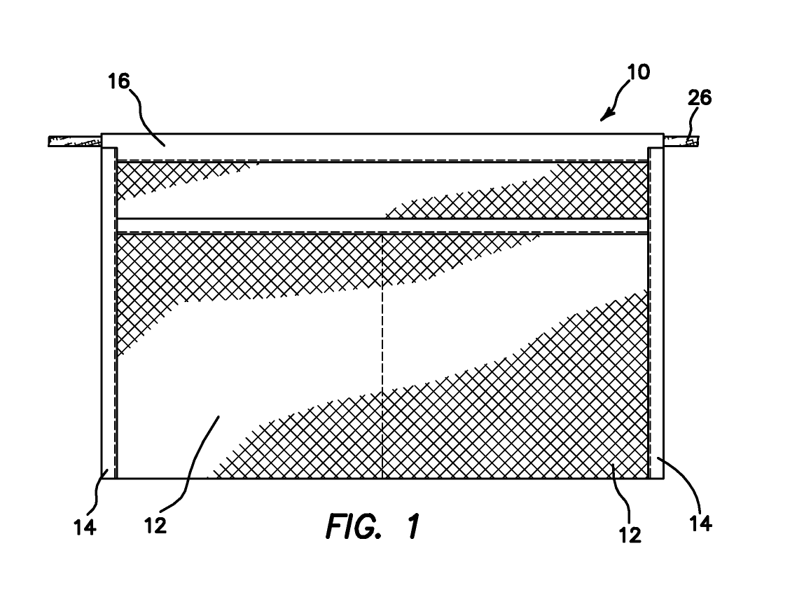

A general embodiment of the current invention is presented in FIG. 1 where the drain pouch, generally denoted by reference numeral 10, may be seen. The drain pouch 10 is substantially rectangular in shape and is comprised principally of a plurality of pockets 12 and a plurality of nylon tapes or nylon bindings, namely at least two lateral nylon tapes 14, at least two pocket nylon tapes 18, and a top nylon tape 16. The plurality of pockets 12 are preferably comprised of light weight, robust, nylon netting that is water resistant, however other materials or fabrics sharing similar characteristics now known or later devised may be used without departing from the original spirit and scope of the invention.

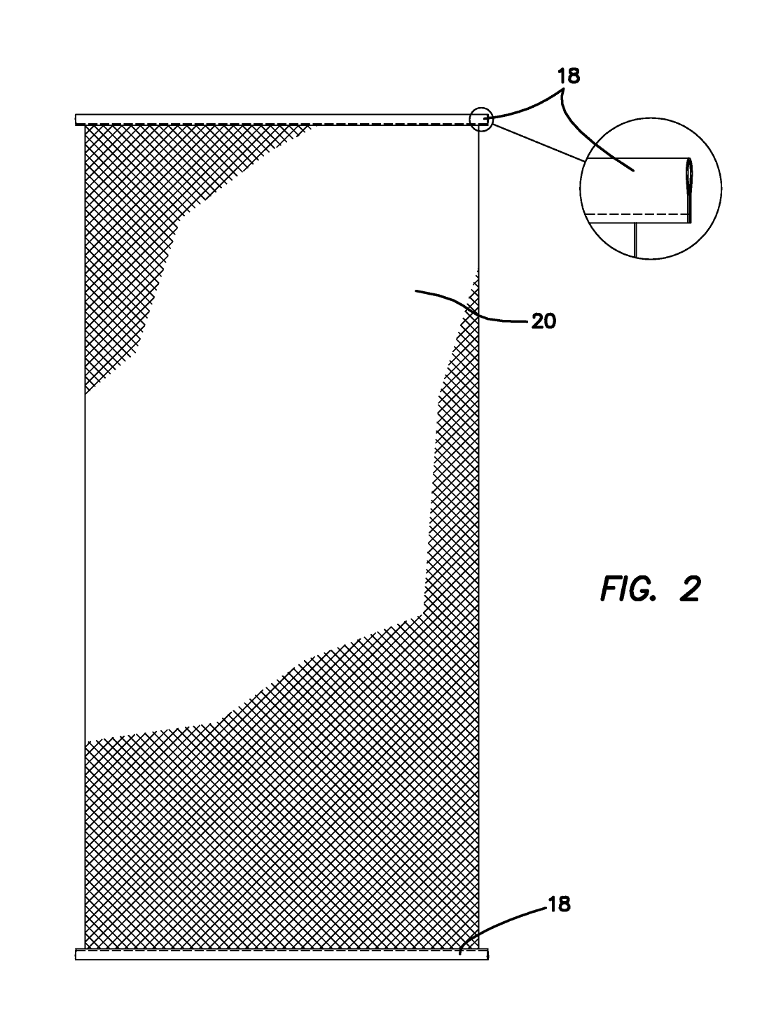

A drain pouch 10 as seen in FIG. 1 may be fabricated by performing the following steps. A rectangle of netting 20 or equivalent material that is preferably 23 inches long and 11.5 inches wide is laid out flat as seen in FIG. 2. A pocket nylon tape 18 is folded over the top and bottom edges of the netting 20. Each pocket nylon tape 18 is folded in half over the top and bottom edges of the netting 20 laterally and then each lateral half is coupled together, binding the netting 20 there between. Each pocket nylon tape 18 is coupled to the netting 20 by sewing stitches, however other means of coupling such as buttons, clasps, glues or adhesives, staples, or their equivalents may be used without departing from the original spirit and scope of the invention.

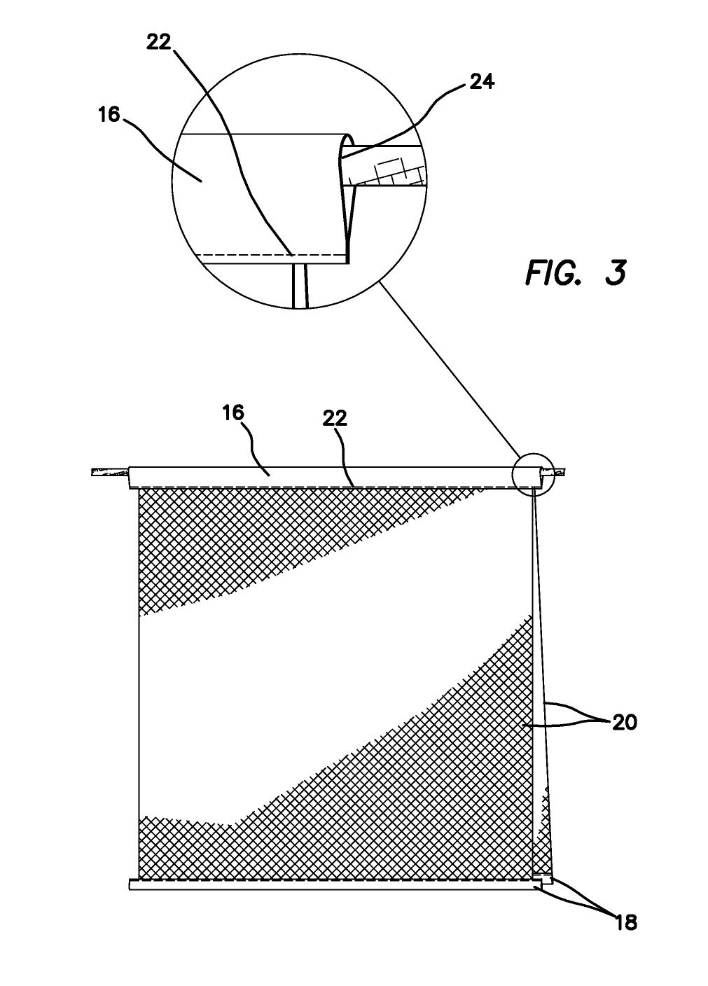

In FIG. 3, the netting 20 has been folded in half, bringing both pocket nylon tapes 18 together side by side. The top nylon tape 16 is then folded laterally over the folded end of the netting 20 with each lateral half then coupled together along a seam 22, trapping the netting 20 there between. The top nylon tape 16 is coupled together along the seam 22 so as to provide an aperture 24 at both ends and an internal hollow passageway defined therein.

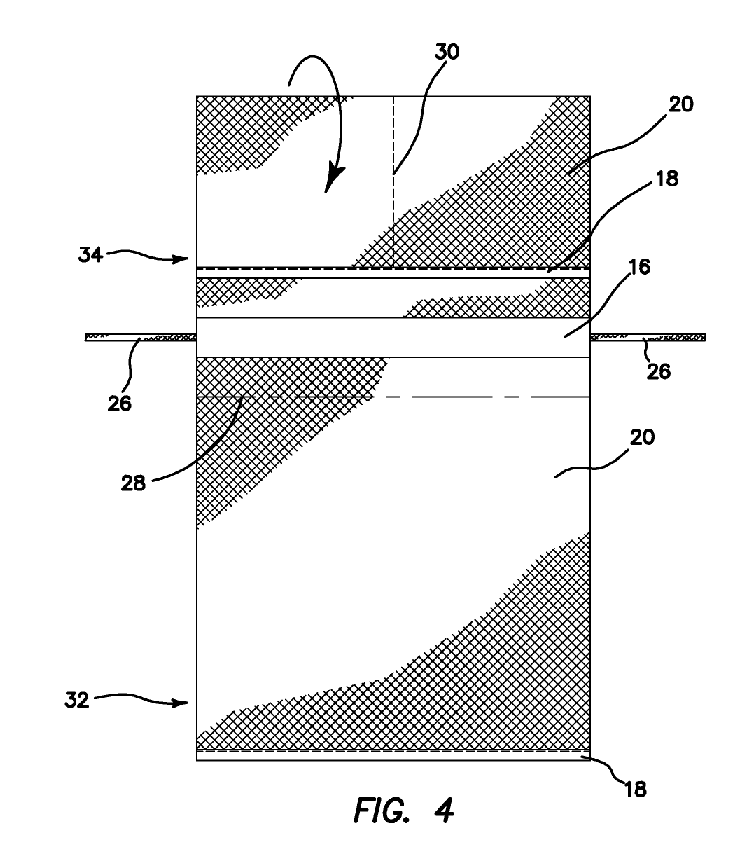

FIG. 4 is top down perspective view of the netting 20 disposed in a butterfly configuration dividing it into a forward portion 32 and a rear portion 34. Here the pocket nylon tapes 18 are folded back towards the top nylon tape 16, namely to a fold line 28 located an inch below the top nylon tape 16 as seen in the forward portion 32 of the netting 20. The pocket nylon tapes 18 are then sewn into position along the fold line 28 as seen in the rear portion 34 of the netting 20, creating a single substantially rectangular shaped pocket. The forward portion 32 and rear portion 34 are then brought together and the two lateral nylon tapes 14 are folded laterally over both edges of the netting 20 and sewn together, trapping the netting 20 there between. Additionally, a pocket division line 30 is sewn down from the pocket nylon tape 18 to the bottom of the netting 20, thus binding the two portions 32, 34 of the pouch 10 together and bifurcating the larger pockets into the four smaller substantially square shaped pockets 12 seen in FIG. 1. The bottom of the pockets 12 are left unaltered so as to provide sufficient flexibility and space for up to four medical drains to be within the drain pouch 10 contemporaneously. Although four pockets 12 are described here and are shown in figures, it is to be expressly understood that fewer or additional pockets 12 within the drain pouch 10 other than what is explicitly shown or described may also be used without departing from the original spirit and scope of the invention.

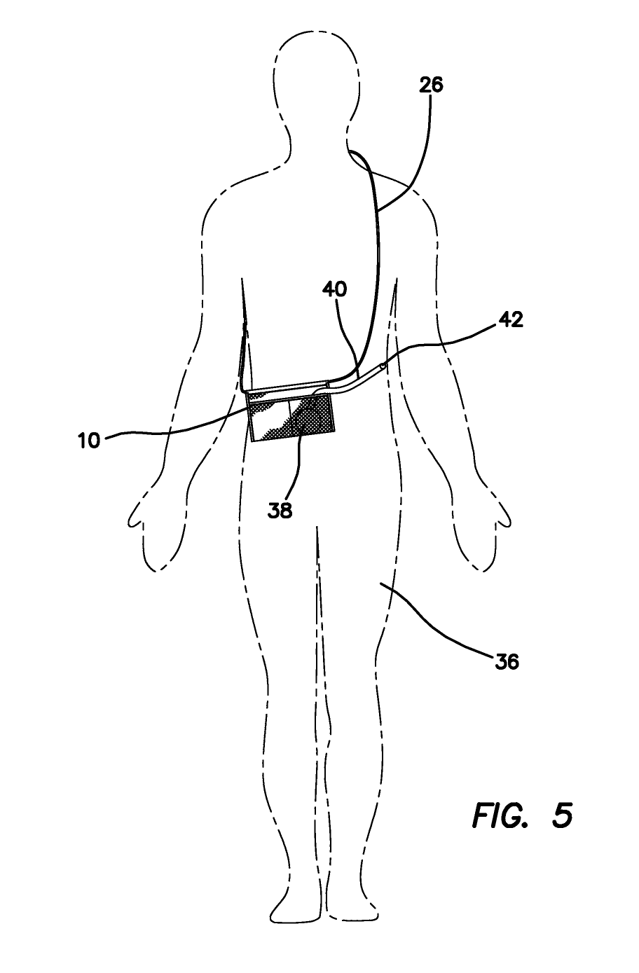

As also seen in FIGS. 1 and 4, a neck cord 26 may be inserted through the apertures 24 defined within the top nylon tape 16 and threaded there through. The neck cord 26 is preferably a loop of soft nylon rope or thread, however other suitable materials now known or later devised such as straps comprised of leather or cloth may also be used within the scope of the invention. The neck cord 26 is long enough so that it may be draped around the neck and/or shoulders of a patient 36 as seen in FIG. 5. The neck cord 26 is also sufficiently long enough so that when a medical drain 38 is disposed within the pouch 10, it rests lower than an insertion site 42. Maintaining the drain 38 below the insertion site 42 at a comfortable position is critical since most drains 38 operate by drawing fluid away from the insertion site 42 through its associated tubing 40 by means of gravity. If the drain 38 is clipped to the patient's 36 clothing or is simply held by the patient 36, the drain 38 may not work effectively and thus cause possible increased discomfort and longer recovery time. Alternatively, the drain pouch 10 may also be placed on a stationary object such as a bed frame, chair, or IV stand for patients who may not be mobile.

Additionally in a related embodiment, the apertures 24 defined within the top nylon tape 16 may be large enough for a typical belt worn by the patient 36 to be threaded there through. After the distal end of a belt is threaded through the top nylon tape 16, it may then be sent through the belt loops of the pants that the patient is wearing as per usual and then into the belt buckle. With the belt firmly coupled around the patient's 36 waist, the drain pouch 10 is then firmly secured at hip level. Regardless if either the neck cord 26 or a belt is threaded through the drain pouch 10, the patient 36 is left unencumbered by the plurality of medical drains 38 that they may be carrying and has both hands available for everyday tasks. Also because the drain pouch 10 comprises a netting 20 that is highly water resistant, the patient 36 may continuously wear the pouch 10 even while in the shower.

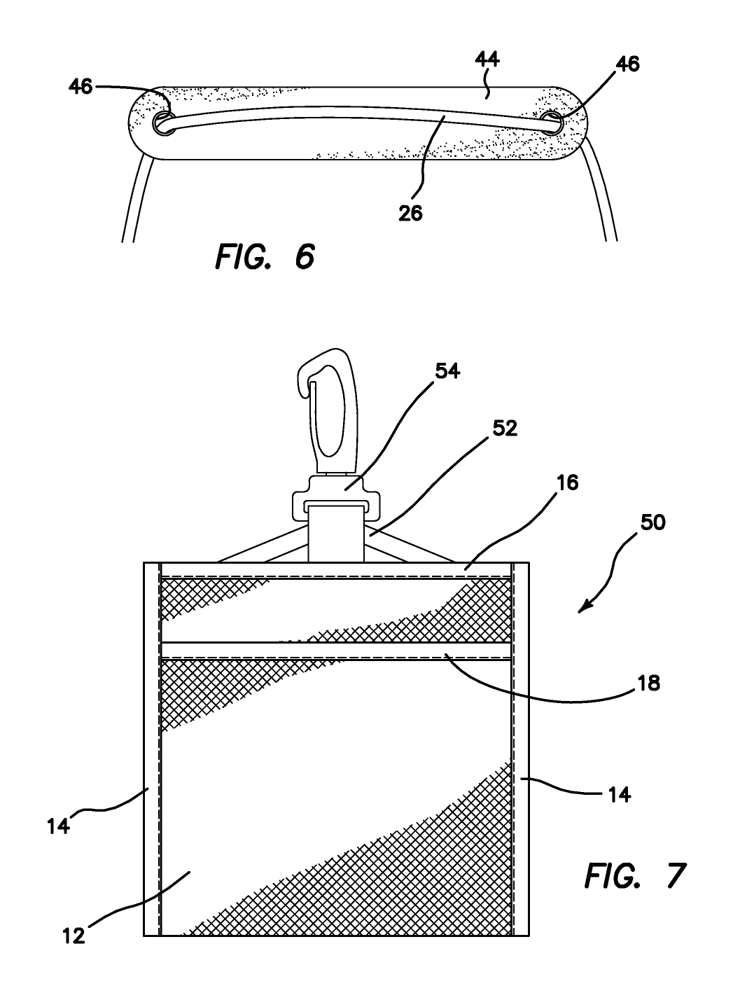

Additionally, the neck cord 26 may comprise a neck protector 44 as seen in FIG. 6. The neck protector 44 is a soft fabric or cloth that comprises at least two eyes 46 defined through its surface. The neck cord 26 is threaded up through one of the eyes 46, disposed across the surface of the neck protector 44, and then threaded down through the opposing eye 46. When the drain pouch 10 is placed around the patient 36, the neck protector 44 acts as a buffer between the skin of the patient and the neck cord 26 and thereby prevents any irritation that may be caused by the neck cord 26 rubbing on the patient's skin over a sustained period of use.

In another embodiment seen in FIG. 7, a smaller drain pouch 50 comprising two pockets 12 disposed back-to-back may also be fabricated using the same steps described above. However in this embodiment, the initial substantially rectangular piece of netting 20 that is used is 5¼ inches wide, or half as wide as the netting 20 used to fabricate the drain pouch 10 seen in FIG. 1. Also seen in FIG. 7, a clip 54 known in the art is coupled to the drain pouch 50 via a plurality of support straps 52 are sewn directly into the top nylon tape 16. The clip 54 may couple to a strap, belt loop or belt buckle, or any other rigid or semi-rigid fixture worn by the patient.

In the acute care or convalescent care hospital it may become necessary for various medical treatment reasons not to connect, drape or apply any pouch straps or belts to the patient's body. For example, when a drain is communicated to a site in the lower portion of the patient's body, such as the lower abdomen, and when the patient spends a substantial amount of time reclining in a hospital bed, it may be necessary to have the drain located at a vertical height below the bed surface or to be otherwise removed from the person of the patient to allow ease of treatment with other types of medical apparatus or procedures. Thus, means other than a body strap or belt for coupling the drain pouch 50 to the patient, the patient's clothing, the bedding or surrounding healthcare apparatus may be used, such as but not limited to alligator clips, Velcro®, suspender clips, double sided tape, hooks and clasps and the like, may be used without departing from the original spirit and scope of the invention.

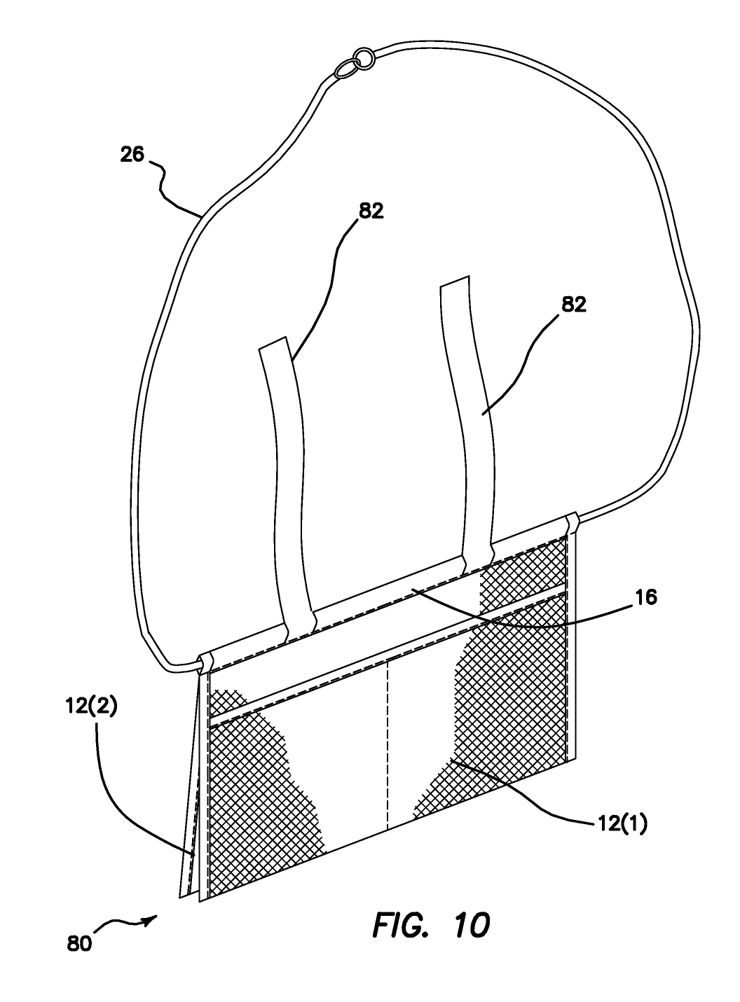

Additionally, in one embodiment, several means for coupling the pouch 50 to the patient may be used contemporaneously. For example, in the pouch 80 seen in FIG. 10 which comprises at least a front pocket 12(1) and at least a back pocket 12(2), both a neck cord 26 and a plurality of straps 82 are coupled to the pouch 80. The neck cord 26 is strung through the top nylon tape 16 as described above while the plurality of straps 82 are coupled directly to the top nylon tape 16 by means of stitches, glue, staples, clasps and the like. The neck cord 26 allows the pouch 80 to be draped about the patient 36 and carried about as described above when the patient 36 is mobile, while the straps 82 which may be comprised of nylon or any other equivalent material, may be tied about a hospital bed rail, IV stand, chair, or other stationary object for when the patient 36 is stationary. In one embodiment, each of the straps 82 comprise a Velcro® tip or other equivalent adhesion means which allows for the straps 82 to be temporarily affixed to one another when wrapped around a fixed or stationary object. When the pouch 80 is tied to a stationary object, a drain 38 may placed in the front pocket 12(1), while the neck cord 26 may be wound up and placed in the back pocket 12(2) for easy access when the patient 36 is to be mobile again.

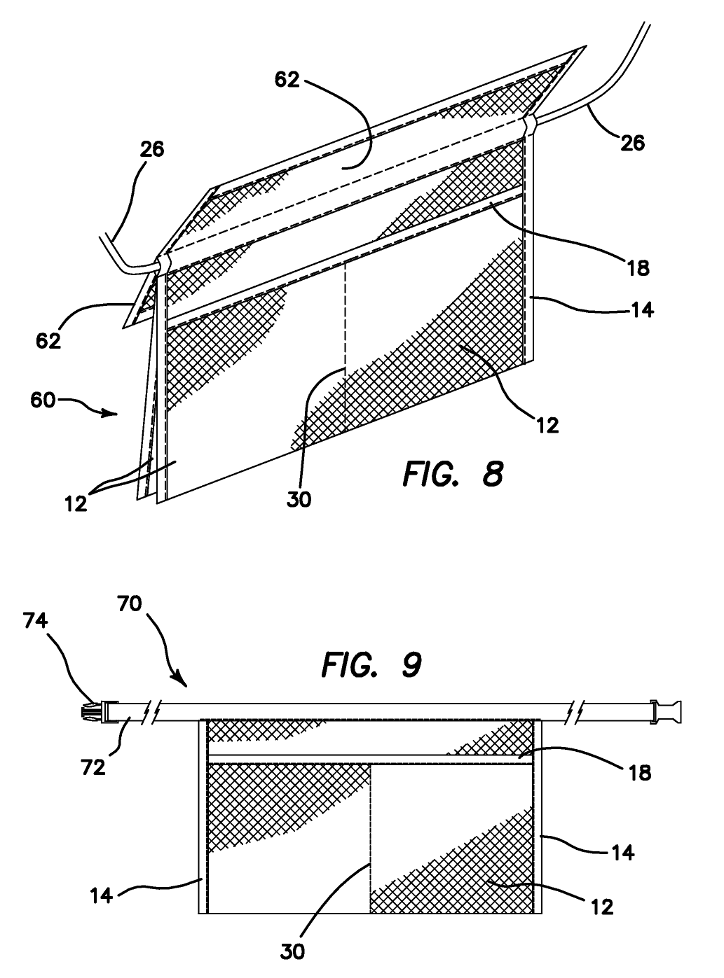

In another embodiment, the drain pouch 10, 50 may be comprised of an opaque material such as canvas or blended fabric in order to hide or conceal the plurality of medical drains 38 that may be placed within the pockets 12. In this embodiment seen in FIG. 8, a drain pouch 60 comprises a pair of flaps 62 disposed on either side of the drain pouch 60. The flaps 62 are sufficiently large enough so that when they are in the down position, they extend down beneath the level of the pocket nylon tape 18 and thus completely conceal any medical drain 38 and a portion of its associated tubing 40 from view. Hiding the medical drains 38 gives the patient 36 a higher sense of normalcy for the duration of their treatment and increases the probability that the patient 36 will comply with their treatment regime by giving them more confidence to enter public areas. Additionally, the opaque drain pouch 60 hides the fluid that is collected by the drains 38, helping those patients who are squeamish or adverse to viewing their own blood and/or other bodily fluids exit their body.

In another embodiment, the pouch is a waist drain pouch 70 as seen in FIG. 9. Here, the waist drain pouch 70 is fabricated in a similar manner to that of the drain pouch 10 seen in FIG. 1, however in this embodiment, a waist band 72 is coupled to the top of the pouch 70 approximately an inch above the pocket nylon tape 18. The waist band 72 is preferably a durable strip of nylon material, however other lightweight and robust fabrics such as leather that are now known or later devised may be used without departing from the original spirit and scope of the invention. Disposed on either end of the waist band 72 is a buckle 74 or other means for adjustably coupling both ends of the band 72 together around the waist of a patient. The buckle 74 may be a typical squeeze-release buckle as shown and as commonly known in the art, however other types buckles, clasps, or ties comprised of metal, plastic, and the like may be used well within the scope of the current invention.

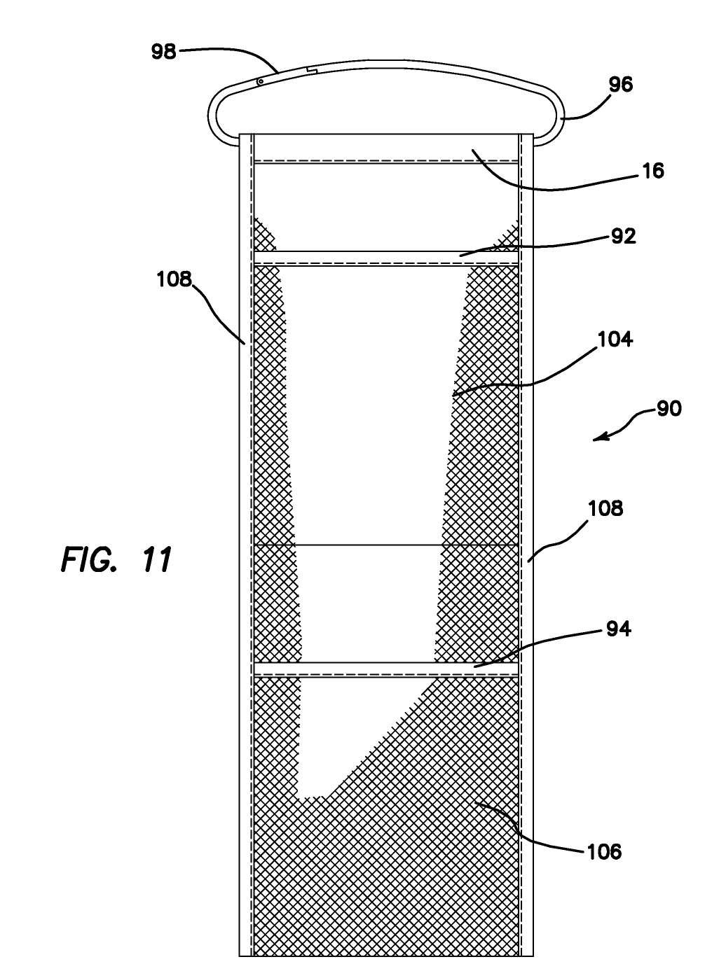

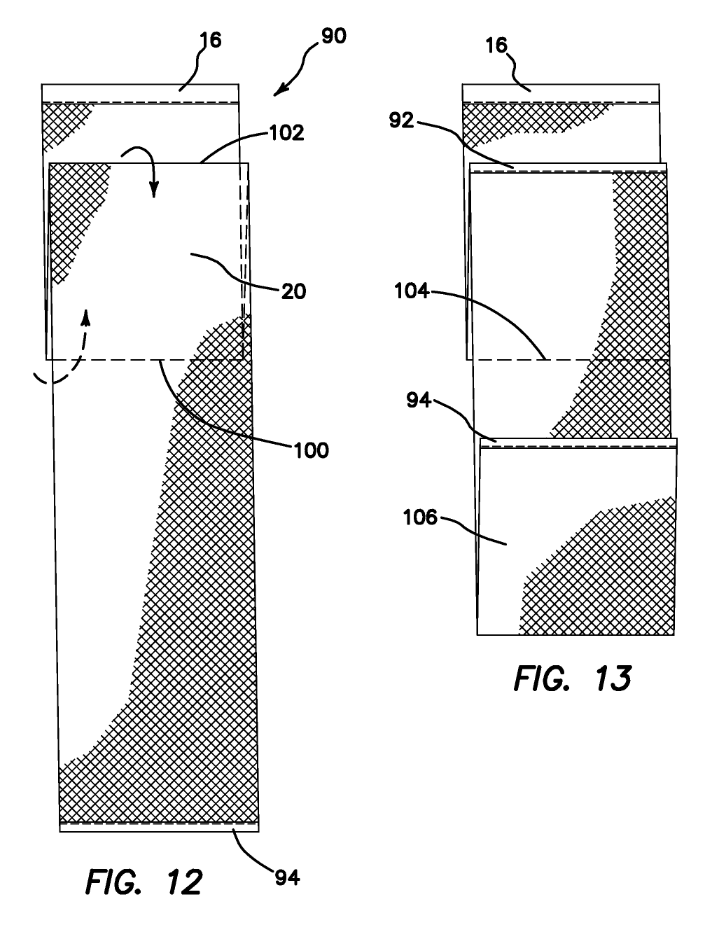

In yet another embodiment, the pouch may be a stacked or tiered pouch 90 as seen in FIG. 11. To fabricate the pouch 90 seen in FIG. 11, a portion a netting 20 similar to that as seen in FIG. 2 with a top nylon tape 16 coupled to one end and a bottom pocket tape 94 coupled to the opposing end is first folded back upon itself at the position of the broken line 100 as seen in FIG. 12. The netting 20 is then folded again back down on itself at line 102. Finally, as seen in FIG. 13, the bottom pocket tape 94 is once again folded back upon itself to form a bottom pocket 106. A top pocket tape 92 is placed over the previous fold line 102 and coupled to the netting 20 to form a top pocket 104. The same process above may then be repeated on the opposing side of the pouch 90, thus giving the pouch 90 up to four pockets for a plurality of drains 38. A pair of extended lateral nylon tapes 108 are then sewn into each lateral side of the pouch 90 as seen in FIG. 11, thus sealing the pockets 104, 106 in on three sides.

A semi-circular ring 96 may then be threaded through the top nylon tape 16 much like the neck cord 26 is threaded there through in the previous embodiment. The ring 96 may be substantially shaped like a half circle or a “D” ring, however other shapes other than what is shown and described here may also be used without departing from the original spirit and scope of the invention. The ring 96 is preferably comprised of hollow plastic tubing, however other equivalent light weight and low cost materials may also be used. The ring 96 also a comprises a spring loaded latch 98 which may be manipulated so as to place the ring 96 about a stationary object such as a hospital bed frame, chair, IV stand, or the like. The substantially semi-circular shape of the ring 96 helps ensure that the pouch 90 and thus any drains 38 disposed therein are centered and straight with respect to the patient 36 no matter what object the ring 96 may be hooked or latched around.

In a further embodiment, the top nylon tape 16 comprises a zipper (not seen). For example, for the drain pouch 10 seen in FIG. 1 or the waist drain pouch 70 seen in FIG. 9, the neck cord 26 or waist band 72 may be disposed across an open and unfolded top nylon tape 16, the top nylon tape 16 comprising a row of zipper teeth along its upper edge and a row of zipper teeth and slider along its lower edge. After the neck cord 26 or waist band 72 has been disposed across the top nylon tape 16, the top nylon tape 16 is folded over the cord 26 or waist band 72, bringing the upper zipper teeth into alignment with the lower zipper teeth. The slider is then manipulated to interlock the zipper teeth together, leaving the neck cord 26 or waist band 72 firmly and securely locked within the top nylon tape 16. The patient 36 may then couple the pouch 10, 70 about their person as discussed above. The same process may also be repeated for any number of coupling means including but not limited to the semi-circular ring 96 seem in FIG. 11 and/or clamps as is known in the art. Other coupling means such as the clip 54 seen in FIG. 7 and the straps 82 seen in FIG. 10 may also be used in conjunction with a zipper by coupling the clip 54 and/or straps 82 to the reverse side of the top nylon tape 16. When the top nylon tape 16 is folded downward and the zipper is engaged, the clip 54 and/or straps 82 are then brought to front of the pouch 50, 80. Additionally, the clip 54 or straps 82 may comprise Velcro® pads at their base or proximal ends so that they may be removably coupled to a corresponding plurality of Velcro® pads disposed on the pouch 50, 80. The zipper may be applied to any of the embodiments disclosed above including but not limited to the stacked or tiered pouch 90 seen in FIG. 11 and the opaque drain pouch 60 seen in FIG. 8.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. A portable drain pouch for holding at least one medical drain comprising:
at least two pockets adjacently disposed for holding the corresponding at least one medical drain;
a plurality of nylon tapes binding each of the at least two pockets on two sides, wherein the bottom of the at least two pockets are not bound and comprise means for expanding the size of the pocket according to the size of the at least one medical drain disposable therein; and
means for coupling the drain pouch to the patient, patient's clothing or object in proximity to the patient.

2. The portable drain pouch of claim 1 where the at least one pocket is comprised of water-resistant netting material.

3. The portable drain pouch of claim 1 where the plurality of nylon tapes binding the at least two pockets on two sides further comprises a top nylon tape coupled to a top edge of the drain pouch.

4. The portable drain pouch of claim 3 where the top nylon tape comprises an aperture and an internal hollow passageway defined therethrough.

5. The portable drain pouch of claim 4 where the means for coupling the drain pouch to the patient comprises a neck cord threaded through the internal passageway and the aperture at both ends of the top nylon tape, wherein the neck cord is adaptable to being formed into a loop and draped over the neck and/or shoulder of the patient.

6. The portable drain pouch of claim 4 where the means for coupling the drain pouch to the patient comprises a belt threaded through the internal passageway and the aperture at both ends of the top nylon tape, wherein the belt is adaptable to being formed into a loop and wrapped around the waist of the patient.

7. The portable drain pouch of claim 3 where the top nylon tape comprises a waist band forming a loop for wrapping around the waist of the patient, and wherein the waist band comprises a buckle for coupling both ends of the waist band together.

8. The portable drain pouch of claim 3 where the means for coupling the drain pouch to the clothing of the patient, patient's clothing or object in proximity to the patient comprises a plurality of clips coupled to the top nylon tape.

9. The portable drain pouch of claim 1 where the at least two pockets are comprised of opaque material.

10. The portable drain pouch of claim 9 further comprising at least one opaque flap disposed over the at least two pockets, wherein the flap conceals a top portion of the pockets when in a down position.

11. A portable apparatus for holding a plurality of medical drains comprising:
a plurality of pockets adjacently coupled to one another in an abutting back-to-back configuration;
a plurality of nylon tapes binding the plurality of pockets on two sides, wherein the bottom of the plurality of pockets are not bound and comprise means for expanding the size of the pockets according to the size of the plurality of medical drains disposable therein; and
means for coupling the drain pouch to a patient, patient's clothing, or object in proximity to the patient.

12. The portable apparatus of claim 11 further comprising where at least two of the plurality of pockets are coupled to one another in a tiered configuration.

13. The portable apparatus of claim 11 where the plurality of nylon tapes binding the plurality of pockets comprises a top nylon tape disposed above the plurality of pockets, wherein the top nylon tape comprises an aperture and an internal hollow passageway defined therethrough.

14. The portable apparatus of claim 13 where the means for coupling the drain pouch to the patient comprises:
a neck cord threaded through the internal passageway and the apertures at both ends of the top nylon tape, wherein the neck cord is adapted to being formed into a loop and draped over the neck, shoulder of the patient, and/or object in proximity to the patient; and
a plurality of straps coupled to at least one of the plurality of nylon tapes, wherein the distal ends of the plurality of straps are adapted to being tied together.

15. The portable apparatus of claim 13 where the means for coupling the drain pouch to the patient comprises a substantially semi-circular shaped ring threaded through the internal passageway and the apertures at both ends of the top nylon tape.

16. The portable apparatus of claim 11 where the plurality of nylon tapes binding the plurality of pockets comprises a top nylon tape disposed above the plurality of pockets, wherein the top nylon tape comprises a waist band forming a loop for wrapping around the waist of the patient, and wherein the waist band comprises a buckle for coupling both ends of the waist band together.

17. A method for accommodating and transporting a plurality of medical drains on or around a patient comprising:
affixing a drain pouch around or to the patient's person;
placing a medical drain within at least one of a plurality of pockets defined in an abutting back-to-back configuration within the drain pouch; and
maintaining the medical drain within the at least one of the plurality of pockets defined within the drain pouch for the duration of the patient's treatment.

18. The method of claim 17 where affixing the drain pouch around or to the patient's person comprises wrapping a neck cord coupled to the drain pouch about the patient's neck and/or shoulders.

19. The method of claim 17 where affixing the drain pouch around or to the patient's person comprises wrapping a waist band coupled to the drain pouch about the patient's waist.

20. The method of claim 17 further comprising concealing the medical drain from view after placing it within the at least one of the plurality of pockets defined within the drain pouch.
